(19) United States
(12) Patent Application Publication
(10) Pub. No.: US 2002/0032154 A1
(43) Pub. Date: Mar. 14, 2002

(54) Interferon-suppressing placental lactogen peptides

(76) Inventor: John A. Peyman, New Haven, CT (US)

Correspondence Address: Dr. John A. Peyman, 336 West Rock Avenue, New Haven, CT, 06515, US

(21) Appl. No.: 09/876,478

(22) Filed: Jun. 7, 2001

Related U.S. Application Data
(60) Provisional application No. 60/210,082, filed on Jun. 7, 2000.

(51) Int. Cl. A61K 38/17; C12N 9/99; C07K 14/435

(52) U.S. Cl. 514/12; 435/184; 530/350

(57) ABSTRACT

Interferon-Suppressing Placental Lactogen Peptides (ISPLP) are disclosed which block actions of the human cytokine interferon-gamma. In addition, methods are disclosed for the treatment with ISPLP of certain disorders associated with increased expression of interferon-gamma-stimulated major histocompatibility complex antigens, such as autoimmune diseases, inflammatory diseases, and transplant rejection.

### CROSS REFERENCES TO RELATED APPLICATIONS

This application claims priority benefit of U.S. application Ser. No. 60/210,082, filed Jun. 7, 2000.

### BACKGROUND OF THE INVENTION

1. Field of the Invention

This invention is directed to Interferon-Suppressing Placental Lactogen Peptides (ISPLP) which can block responses to the human cytokine interferon-gamma. Specifically, this invention relates to the use of ISPLP in the treatment of certain disorders associated with increased expression of interferon-gamma-stimulated major histocompatibility complex antigens, such as autoimmune diseases, inflammatory diseases, and transplant rejection.

2. Description of the Related Art

Fetal-maternal Immunology and Unresponsiveness of Trophoblasts to Interferon-gamma.

Many vertebrates bear live young, but only in eutherian (placental) mammals were the immunological problems associated with increased hematogenous delivery of nutrition to the embryo solved by the evolution of the trophoblast (Gerhart, J., and Kirschner, M. 1997. Cells, embryos, and evolution: Toward a cellular and developmental understanding of phenotypic variation and evolutionary adaptability. Blackwell Science (Malden,Mass.)). The absence of polymorphic major histocompatibility class I and class II molecules on trophoblasts in the normal human placenta is thought to be a critical factor in the survival of the semi-allogeneic fetus and placenta. Syncytiotrophoblast covering the chorionic villi forms not only a large transport surface for efficient gas, nutrient, and waste product exchange between the maternal and the fetal blood supplies but also a non-immunogenic, mechanical barrier that excludes maternal blood cells from the fetal circulation (Hunt, J S, and Orr, H T (1992) FASEB J 6: 2344-2348 “HLA and maternal-fetal recognition”; Cross J C, Werb Z, and Fisher S J (1994) Science 266: 1508-1518 “Implantation and the placenta: Key pieces of the development puzzle”; Wood G W (1994) Immunol. Today 15: 15-18 “Is antigen presentation the explanation for fetal allograft survival?”). The induction of expression of MHC class I and II genes occurs during a normal immune response in cells other than placental trophoblasts and is stimulated by any one of several cytokines, including interferon-gamma (Ting J P and Zhu X S (1999) Microbes Infect 1:855-61 “Class II MHC genes: a model gene regulatory system with great biologic consequences.”; van den Elsen P J, Gobin S J, van Eggermond M C, and Peijnenburg (1998) Immunogenetics 48:208-21 “Regulation of MHC class I and II gene transcription: differences and similarities.”). A cytoplasmic factor, TSU, expressed in trophoblasts and a cytokine, TGF-beta, secreted by trophoblasts have been shown to contribute to the repression of MHC class II genes (Peyman J A (1999) Biol Reprod 60: 23-31, “Repression of major histocompatibility complex genes by a human trophoblast ribonucleic acid”; Piskurich J F et al. (1999) Mol Cell Biol 19:431-440 “Two distinct gamma interferon-inducible promoters of the major histocompatibility complex class II transactivator gene are differentially regulated by STAT1, interferon regulatory factor 1, and transforming growth factor β”). The invention adds a third, independent factor to these two physiological mediators, the full-length peptide of the invention is known to be a major secretion.product of trophoblasts. Identification of this novel function for the N-terminal peptide of hPL provides the opportunity to recapitulate therapeutically part of the immunosuppressed phenotype of placenta, while avoiding the somatotropic and mammotropic effects which reside in a larger combination of polypeptide regions of the native hormone.

Exacerbation of Autoimmunity, Inflammation, and Transplant Rejection by IFN-gamma.

A large body of evidence suggests that IFN-gamma-stimulated expression is responsible for major histocompatibility complex (MHC) associated autoimmune diseases. For example, elevated serum levels of IFN-gamma. correlated with autoimmunity (Hooks et al. (1979), New England J Med 301: 5-8). Aberrant MHC gene product expression correlated with some forms of autoimmunity (Battazzo et al. (1983) Lancet 1115-1119). Higher IFN-gamma. levels correlated to greater severity of disease in SLE patients, and histamine-release enhancing activity of interferon can be inhibited by anti-interferon sera. (Hooks et al. (1980) Ann NY Acad Sci 21-32). Anti-IFN-gamma monoclonal antibody eliminated the ability of leucoagglutinin-stimulated T cells to induce HLA-DR expression (Iwatani et al (1986) J Clin Endocrin and Metabol 63:695-708). It is hypothesized that excess IFN-gamma causes the inappropriate expression of MHC gene which, in turn, causes autoimmune reactions against the tissues whose cells are inappropriately expressing the MHC proteins and displaying autoantigens bound to the MHC proteins.

In view of the above, it would be advantageous to have available agents that could suppress the action of IFN-gamma, as required for therapy. Such agents would be highly advantageous for treatment of diseases associated with inappropriate or inadequate immune responses, such as autoimmune diseases, inflammatory diseases, and transplant rejection.

Interactions between T lymphocytes and Interferon-gamma-Stimulated Antigen-Presenting Cells.

In stimulating immune responses, antigens elicit many molecular and cellular changes. Lymphocytes recognize antigens as foreign and are responsible for initiating both cellular and humoral responses against the presenting antigen. B lymphocyte cells respond to antigen by the production of antibodies against the presenting antigen; T lymphocytes respond by initiating a cellular response to the presenting antigen. The two major subsets of T cells are T-helper cells, involved in processing of antigen for presentation to B cells, characterized by the presence of a cell-surface glycoprotein called CD4, and cytolytic T lymphocytes (CTLs), involved in recognition of antigen on cell surfaces and lysis of cells recognized as foreign, characterized by the presence of a cell-surface glycoprotein called CD8. T cells recognize peptide fragments in conjunction with one of the two main classes of cell-surface glycoproteins of the major histocompatibility complex (MHC): either class I (MHC-I) or class II (MHC-II) proteins. CD8+ T cells recognize antigens in conjunction with MHC-I, whereas CD4+ T cells recognize them in conjunction with MHC-II.

The MHC Class I genes encode the principal subunits of MHC-I glycoproteins, called human leukocyte antigens in humans, the principle ones being HLA-A, B, and C. These are present on virtually all cells and play a major role in rejection of allografts. They also form complexes with peptide fragments of viral antigens on virus-infected cells: recognition of the complexes by CD8+ CTLs results in destruction of virus infected cells. Recognition of the complexes is by a single receptor on the T cells which recognizes antigen in combination with MHC. MHC class I antigens are expressed constitutively at low or moderate levels on essentially all nucleated cell types, and high-level expression is induced in cells and tissues under the influence of pro-inflammatory cytokines such as interferon-gamma.

MHC Class II genes, the major classes in humans being known as DP, DQ (subclasses beta2, alpha2, and beta1, alpha1) and DR (subclasses beta1, beta2, beta3 and alpha1), encode cell-surface glycoproteins that are expressed by professional antigen-presenting cells, principally B cells, macrophages and dendritic cells. However, most human cell types except trophoblasts can be induced to express high levels of MHC class II antigens following interferon-gamma stimulation and then function to some extent as antigen-presenting cells. Together with peptide fragments of antigen, the class II proteins from the epitopes that are recognized by T helper cells (CD4+).

T lymphocyte (T-cell) mediated immune reactions can be organized into three sequential activation steps. First, CD4+ and CD8+ T-cells recognize the presence of autologous MHC class II and class I proteins, respectively, on the surface of a cytokine-stimulated tissue cell presenting an autoantigen in the case of autoimmune disease or the presence of non-autologous MHC class II and class I proteins, respectively, on the surface of an cytokine-stimulated foreign cell, in the case of allograft rejection.

Second, the T-cells are activated by interaction of a ligand with the T cell receptors and other accessory stimulatory molecules which are dependent also on cytokines such as interferon-gamma for stimulation of high level expression on antigen-presenting cells (APC). Most important is the interaction between the antigen specific T cell receptor and ligand, a complex of MHC and antigenic peptide on the antigen presenting cell. Other receptors present on the T cell must also be contacted by their ligands on APC, such as the cell adhesion molecule ICAM-1 and the co-stimulatory molecules B7-1 and B7-2, to insure activation. Once activated, the T-cells synthesize and secrete interleukin-2 (IL-2) and other cytokines.

The cytokines secreted by the activated T-cells, including interferon-gamma, lead to the third, or effector, phase of the immune response which involves recruitment and activation of lymphocytes, monocytes, and other leukocytes which together lead to cell lysis, as reviewed, for example, by Pober (Pober et al. (1990) Human Immunol. 28:258-262 “The potential roles of vascular endothelium in immune reactions”).

Several therapeutics, such as cyclosporin A, block the activation of T-cells. Other attempts, however, to interrupt T-cell-APC interactions have generally met with limited success. For example, several strategies have tried to use reagents of various types, including antibodies and blocking proteins, to interfere with adhesion between T-cells and foreign cells. T-cell vaccines have been used (Lider et al., (1988) Science 239:181 “Anti-idiotypic network induced by T cell vaccination against experimental autoimmune encephalomyelitis”). T-cell receptor blocking antibodies can reduce symptoms in animal models (Owhashiand et al. (1988) J Exp Med 168:2153 “Protection from experimental allergic encephalomyelitis conferred by a monoclonal antibody directed against a shared idiotype on rat T cell receptors specific for myelin basic protein”). Antibodies to CD4 can block the activity of T-helper cells (Brostoffand et al. (1984) J Immunol 133:1938 “Experimental allergic encephalomyelitis: successful treatment in vivo with a monoclonal antibody that recognizes T helper cells”), and blocking peptides that occupy T-cell receptors have been developed (Adorini et al. (1988) Nature 334:623-628 “Dissociation of phosphoinositide hydrolysis and Ca2+ fluxes from the biological responses of a T-cell hybridoma”). These strategies have generally resulted in immune responses to the reagents, rather than the desired interruption of T-cell-APC binding.

Suppression of IFN-gamma-induced expression of a number of important immunostimulatory molecules, such as the MHC and cell adhesion components, on cytokine-activated tissue cells functioning as initiators of immune reactions would be a useful adjunct to current therapies used for reduction of inappropriate immune responses in various human diseases and conditions. It is a goal of the present invention to define said therapeutic immunosuppressants.

Somatogenic and Lactogenic Activities of hPL, hGH, and hPRL.

The human growth hormone/human placental lactogen (hGH/hPL) gene cluster on chromosome 17q22-q24 contains five genes: hGH-1, hGH-V, hPL-3, hPL-4, and hPL-1 (Chen E Y et al. (1989) Genomics 4 (4), 479-497 “The human growth hormone locus: nucleotide sequence, biology, and evolution”). The expression and the functions of the first four genes have been well documented. hGH-1 is produced by the pituitary. The hGH-V gene is expressed in syncytiotrophoblasts of placenta. The hPL-3 and hPL-4 genes encode identical placental lactogen polypeptides that are abundantly expressed by placenta. In contrast, the hPL-1 gene was originally considered a pseudogene inactivated by loss of the normal intron 2 splice donor site. However, hPL-1 transcripts are present in human placenta and that their levels are increased during the second trimester, and it is now known that about 25% of the hPL-1 gene product is secreted hPL and the remainder consists of non-secreted polypeptides. (Misra-Press et al. (1994) J. Biol Chem 269:23220-23229 “Complex alternative splicing partially inactivates the human chorionic somatomammotropin-like (hCS-L) gene”). The physiological significance of this minimally functional hPL gene was inferred in several genetic studies of infants with deletions in the hGH/hPL locus in which only the hPL-1 gene remained intact. No abnormalities of intrauterine growth or development were observed, although the newborns displayed the phenotype of isolated hGH deficiency. (Gossens et al. (1986) J. Clin Endo Metab 62: 712-716; Wurzel et al. (1982) DNA 1: 251-257). Finally, three placental lactogen genes are conserved in the rhesus monkey and encode proteins represented by full-length cDNAs, consistent with a fully functional hPL-1 gene in the recent evolutionary past (Golos T G et al. (1993) Endocrinology 133:1744-52 “Cloning of four growth hormone/chorionic somatomammotropin-related complementary deoxyribonucleic acids differentially expressed during pregnancy in the rhesus monkey placenta”). For clarity sake, Table 1 is provided which lists synonyms of each of the members of the hGH/hPL family of polypeptides. Rarely used names are in parenthesis. The hPL-3 and hPL-4 genes encode the same mature polypeptide, herein referred to simply as hPL.

TABLE 1
Summary of nomenclature of hGH/hPL genes and proteins.

| Human gene | | | |
|---|---|---|---|
| or hormone | Synonyms | | |
| Growth hormone | Somatotropin | | |
| hGH-1 | hGH-N | | |
| hGH-V | hGH-2 | (hPL-2) | |
| Placental lactogen | Chorionic somatomammotropin | | |
| hPL-3 | hCS-1 | hCS-A | hPL (herein) |
| hPL-4 | hCS-2 | hCS-B | hPL (herein) |
| (hPL-1) (pseudogene) | hCS-5 | hCS-L | hPL-1 (herein) |

Human prolactin (hPRL) is a member of a separate protein family, the prolactin family. The hGH family has been characterized in considerable detail as to their growth promoting activities and binding to the growth hormone receptor and to the prolactin receptor. Full-length hPL shares 85% sequence identity to hGH yet has some very different receptor-binding properties. For example, hPL binds 2300-fold weaker than hGH to the hGH receptor, yet these two hormones have similar affinities for prolactin receptors. As with hGH, mutation of Glu-174 to Ala in hPL reduces the affinity for the hPRL receptor by 1400-fold. The affinity of hPL can be increased by over 200-fold for the hGH receptor by installing four hGH receptor determinants that are not conserved in hPL. By simultaneously introducing E174A, a pentamutant is produced whose binding affinity for the hGH receptor is only 1.6-fold weaker than hGH, but whose binding affinity for the hPRL receptor is weaker by greater than 1000-fold relative to wild-type hPL. Thus, an hPRL receptor-binding epitope can be defined in hPL, an hGH receptor-binding epitope can be “recruited” into hPL, and receptor selective analogs of hPL can be produced that are designed to bind tightly to either, neither, or both receptors. Such variants help understand specific receptor-binding, activation, and subsequent signaling events of full-length hPL and full-length variants (Lowman et al.(1991) J Biol Chem 266:10982-10988 “Mutational analysis and protein engineering of receptor-binding determinants in human placental lactogen”).

The 28-residue N-terminal regions of hGH and hPL are known to form, with additional distal regions, a part of the tripartite hormone-receptor interface in the interaction of full-length hGH and hPL and the hGH receptor, but this N-terminal region by itself is inactive in hGH receptor binding. A number of studies have used the homolog-scanning mutagenesis technique, in which gene fragments of hPL or hPRL are systematically substituted throughout the hGH gene, thus producing various chimeric hormones. These studies and others have shown that the N-termini of PL, GH, and PRL in several species are required for binding to the prolactin receptor and growth hormone receptor, but are not sufficient for binding with high affinity to growth hormone receptor (Cunningham B C and Wells J A (1989) Science 244: 1081-1085 “High-resolution epitope mapping of hGH-receptor interactions by alanine-scanning mutagenesis”; Luck D N et al. (1989) Mol Endocrinol 3: 822-831 “Bioactive recombinant methionyl bovine prolactin: structure-function studies using site-specific mutagenesis”; Gertler A (1992) J Biol Chem 267: 12655-12659 “Preparation, purification, and determination of the biological activities of 12 N terminus-truncated recombinant analogues of bovine placental lactogen”; Strasburger CJ et al. (1989) Mol Cell Endocrinol 67: 55-62, “Indication of different lactogen and somatogen binding sites in the human growth hormone molecule as probed with monoclonal antibodies”). These results are consistent with the idea that the native hPL hormone carries out its previously characterized somatotropic and lactogenic functions by means of several non-contiguous structural regions closely related to full-length growth hormone and full-length prolactin, but carries out the novel MHC suppression function through binding solely of the N-terminal region to either prolactin receptor or an uncharacterized hPL receptor.

Physiological Functions of Full-length hPL(1-191).

hPL is a placental hormone that has been studied as a modulator of maternal metabolism during pregnancy, as a stimulator of mammogenesis, lactation, and maternal behavior, and as a significant stimulator of fetal growth (Goffin V et al. (1996) Endocrine Rev 17: 385-410 “Sequence-function relationships within the expanding family of prolactin, growth hormone, placental lactogen, and related proteins in mammals”; Forsyth I A (1994) Exp Clin Endocrinol 102: 244-251 “Comparative aspects of placental lactogens: structure and function”; Byatt J C et al. (1992) J Animal Science 70: 2911-2913; Byatt J C et al. (1994) J Endocrinol 140: 33-43 “Stimulation of mammogenesis and lactogenesis by recombinant bovine placental lactogen in steroid-primed dairy heifers”; Bridges R S and Freemark M S (1995) Hormones & Behavior 29: 216-226 “Human placental lactogen infusions into the medial preoptic area stimulate maternal behavior in steroid-primed, nulliparous female rats”; Freemark M et al. (1989) Endocrinol 125:1504-1512 “Nutritional regulation of the placental lactogen receptor in fetal liver: implications for fetal metabolism and growth”).

Knockout mice often provide useful genetic information, but in an analysis of the necessity for the placental hGH/hPL hormones during pregnancy, the recourse of using such mice is not available since in rodents the placental lactogen genes are members of a prolactin gene family of about 15 members including prolactin-like proteins, prolactin-related proteins and proliferins. This family of rodent hormones itself has interesting and complex functions in growth and development which unfortunately can not help explain the functions of the human hGH/hPL hormones (Soares M J et al. (1998) Biol Reprod 58: 273-284 “The uteroplacental prolactin family and pregnancy”).

Reduced maternal blood levels of hPL have been correlated in many studies of pregnancies complicated by arterial hypertension or by intrauterine growth retardation, but no causal relationship has been established. Significantly, the specific lack in abnormal pregnancies of hPL-positive trophoblast cells that normally infiltrate the endometrial spiral arteries (Gosseye S and van der Veen F (1992) Eur J Obst Gyn Reprod Biol 44: 85-90 “HPL-positive infiltrating trophoblastic cells in normal and abnormal pregnancy”), and the lack of hPL-positive mature trophoblast at the implantation site in pre-eclampsia (Redline R W, Patterson P (1995) Hum Pathol 26: 594-600 “Pre-eclampsia is associated with an excess of proliferative immature intermediate trophoblast”) are consistent with a functional role for hPL in MHC suppression during normal blastocyst implantation. However, no previous studies have formally shown a negative correlation between hPL and MHC gene expression.

Anti-mitogenic Effects of hPL on Phytohemagglutinin-stimulated Human T-lymphocytes and Lipopolysaccharide-stimulated Human B-lymphocytes.

Experiments in the 1970's on the effects of unfractionated pregnancy-serum or unfractionated pregnancy-lymphocyte populations gave a spectrum of outcomes depending on the experimental model of immunity used. The question of fetal-maternal tolerance has remained largely unanswered until recently. Several early immunological studies succeeded in demonstrating that exogenous full-length hPL reduced the peripheral blood T-lymphocyte mitogenic response to treatment with phytohemagglutinin in vitro or the tonsil B-lymphocyte mitogenic response to treatment with lipopolysaccharide in vitro (Contractor S F and Davies H (1973) Nature 243: 284 “Effect of human chorionic somatomammotropin and human chorionic gonadotrophin on phytohemagglutinin-induced lymphocyte transformation”; Cerni C et al. (1977) Arch Gynak 223: 1-7 “Immunosuppression by human placental lactogen (HPL) and the pregnancy-specific beta1-glycoprotein (SP-1)”; Hammerstrom L. et al. (1979) Acta Obstet Gynecol Scand 58: 417-422 “The immunodepressive effect of human glucoproteins and their possible role in the nonrejection process during pregnancy”). Several other pregnancy hormones have shown similar immunosuppressive activities when measured in lymphocyte activation assays in vitro, including progesterone, estrogen, and human chorionic gonadotrophin. Those early studies on suppression of lymphocyte activation by hPL have not been revisited with current technology. While numerous studies have looked into changes that occur in the immune system during pregnancy, no results have appeared on the effects of full-length hPL or hGH or their N-terminal fragments on the expression of major histocompatibility complex proteins in cytokine-activated antigen-presenting cells or professional antigen presenting cells.

Hypoglycemic Function of an Isolated Peptide from the N-terminal Region of hGH.

An octapeptide representing hGH(6-13) was shown to have hypoglycemic activity which was determined by an intravenous insulin tolerance test. The hGH(6-13) had no effect on growth (Ng FM et al. (1974) Diabetes 23:943-949). The hGH peptide augmented insulin secretion in a glucose-dependent manner, and increased binding of insulin to insulin receptor on isolated cells (Ng, FM (1988) Diabetes Res Clin Pract 19:17-24 “A comparison of cellular actions between gliclazide and a hypoglycaemic peptide fragment of human growth hormone”). While this hGH(6-13) peptide is in a conserved region in the hGH/hPL polypeptide family and contains 7 of 8 residues (88%) identical to an homologous hPL octapeptide, there is no information on the regulation of interferon gamma-stimulated genes by the hGH(6-13) octapeptide and there is no description of the preparation or analysis of an hPL(6-13) octapeptide.

Anti-angiogenic Activities of N-terminal Fragments of hGH(1-150), hPL(1-150), hGH-V(1-150), and hPRL(1-150).

N-terminal fragments of size 16-kDa (residues approximately 1-150) which were prepared in vitro from hPL, hGH, hGH-V, and hPRL were shown to suppress the angiogenic activity of fibroblast growth factor in a bovine brain capillary endothelial cell (BBCE) assay All four 16-kDa peptides had a dose-dependent inhibitory effect on basic fibroblast growth factor (bFGF)-induced BBCE cell proliferation. The intact hormones, hGH and hGH-V, on the other hand, over-stimulated the bFGF-induced cell proliferation up to a maximum of 2-fold the level obtained with bFGF alone. Intact hPL and hPRL had no significant additive somatotropic effect in the BBCE assay. In an in vivo chick chorioallantoic membrane (CAM) assay the activity of the intact molecules and fragments was examined. The CAM appears in the yolk sac at 48 h, grows rapidly over the next 6-8 days, and stops growing on day 10. An early-stage CAM bioassay (days 6-8) was performed to assess the effects on developing capillaries, and a late-stage bioassay (days 10-14) was performed to test the effects on non-growing quiescent CAM. In the early-stage CAM assay, an avascular area was clearly present surrounding the disks containing hPL, hGH, hGH-V, or hPRL 16-kDa fragments. The corresponding full length hormones had no effect in this bioassay. In the late-stage bioassay, the intact proteins stimulated new capillary and blood vessel formation, which could be observed emerging from the protein-containing disks whereas the 16-kDa fragments had no effect (Struman I et al. (1999) Proc Natl Acad Sci USA 96: 246-1251 “Opposing actions of intact and N-terminal fragments of the human prolactin/growth hormone family members on angiogenesis: An efficient mechanism for the regulation of angiogenesis”).

The polypeptide sequences effective in anti-angiogenic activity include the N-terminal 28-residues of hPRL. This sequence is not homologous to the corresponding N-terminal 28-residue sequences of the 5 members of the hGH/hPL family, and hPRL(1-28) shares 11% identity, for example, with hPL(1-28). This negative correlation indicates that the sequence motifs in hPL that suppress several effects of interferon, as defined by the present invention, are distinct from the sequence motifs spanning the N-terminal region and additional regions of the corresponding 16-kDa fragments of the hGH, hPL, and hPRL polypeptides that are active in anti-angiogenesis.

### BRIEF SUMMARY OF THE INVENTION

The present invention relates to Interferon-Suppressing Placental Lactogen Peptides (ISPLP) with substantial sequence identity to the N-terminal 28 residues of hPL, and derivatives thereof. The invention further pertains to therapeutic uses of such ISPLP, and especially hPL(1-28), for the treatment of autoimmune diseases, inflammatory diseases, and transplant rejection.

In detail, the invention pertains to peptide fragments which suppress the actions of interferon-gamma (IFN-gamma) and which are derivable from naturally occurring amino acid sequences.

The invention comprises an ISPLP selected from the group consisting of:

(A) an isolated hPL(1-28) peptide comprising the sequence:

VQTVPLSRLFDHAMLQAHRAHQLAIDTY (SEQ ID NO:4), or a derivative thereof,

(B) an isolated hPL-1(1-28) peptide comprising the sequence:

VQTVPLSRLFKEAMLQAHRAHQLAIDTY (SEQ ID NO:7), or a derivative thereof, wherein the peptide suppresses the action of IFN-gamma.

The invention further pertains to a method for suppressing the action of IFN-gamma which comprises providing to a human cell or tissue an effective amount of the ISPLP disclosed above.

The invention further pertains to a method for treating a human disorder selected from the group consisting of:

(A) autoimmune diseases,

(B) inflammatory diseases, and

(C) organ transplant rejection,

which comprises administration of an effective amount of the ISPLP discussed above to a subject in need of such treatment.

### BRIEF DESCRIPTION OF THE FIGURES

FIG. 1. Alignment of the N-termini of the gene products of three hPL genes (SEQ ID NOS. 2, 3 and 6). Conserved residues in the secreted polypeptides are shown in bold letters. A total of 26 amino acid residues (93%) are conserved in the N-terminal 28 amino acids of the 3 secreted placental lactogen gene products. The stop codon in hPL(1-28) is indicated by an asterisk. The three dots following each sequence indicate the continuation which is not shown of the polypeptide sequence in the natural proteins.

FIG. 2. Alignment of hPL(1-28) (SEQ ID NO:2) with hPL-3 (also SEQ NO.2) and hPL-4 (SEQ ID NO:3). The stop codon in hPL(1-28) is indicated by an asterisk. The secreted peptide of the invention is identical (100%) to the N-terminal 28 amino acid residues of the 2 major hPL gene products.

FIG. 3. Alignment of hPL(1-28) with hPL-1(1-28) (SEQ ID NO:6). The peptide of the invention contains 26 residues identical (93%) to the minor hPL gene product. The 2 non-identical residues are not similar, according to their charge in solution at neutral pH.

FIG. 4. Alignment of hPL(1-28) with hGH-1 (SEQ ID NO:14). The peptide of the invention contains 20 residues identical (71%) to the putuitary hGH gene product. A total of 5 of the 8 non-identical residues are similar in hydrophobicity and charge, resulting in an overall similarity of 89%.

FIG. 5. Alignment of hPL(1-28) with hGH-V(1-30) (SEQ ID NO:15). The peptide of the invention contains 13 residues identical (46%) to the placental hGH gene product. The 6 similar residues raise the overall similarity to 68%.

FIG. 6. Alignment of hPL(1-28) with HPRL(1-28) (SEQ ID NO:16). The peptide of the invention contains only 3 residues identical (11%) and 12 residues similar (43%) to prolactin.

FIG. 7. Use of partially overlapping oligodeoxynucleotides to construct a peptide-expression cassette. FIG. 7A, hPL(1-28) peptide can be prepared by annealing at their respective 3′-ends as shown, two oligonucleotides (SEQ ID NOS. 9 and 10), for subsequent extension with a Taq DNA polymerase followed by cloning into a suitable, eukaryotic, expression vector. The protein encoded extends from the start methionine and signal peptide through the secreted peptide to the stop codon. FIG. 7B, DNA used similarly to produce secreted hPL-1(1-28).

FIG. 8. Schematic diagram of expression cloning method leading to the identification of the interferon-gamma suppressor hPL(1-28) peptide.

FIG. 9. Sequence of hPL(1-28) cDNA and encoded polypeptide. FIG. 9A, The signal peptide is enclosed in a box, and the secreted peptide is labeled at residues 1 and 28. FIG. 9B, cDNA sequence (SEQ ID NO.1) and open reading frame ending with a termination codon (Ter) after Tyrosine-28.

FIG. 10. Suppression of interferon-responsive antigens by hPL(1-28) in stably transfected HeLa cells. Flow cytometric analysis of constitutive and IFN-gamma-inducible cell surface expression of MHC class II antigens, MHC class I antigens, and ICAM-1 antigen. mAb used in each case is indicated above each column. Doses of IFN-gamma are noted on the right side.

FIG. 11. Suppression of interferon action by transfected hPL(1-28). Quantitation of the flow cytometric results in FIG. 10. FIG. 11A, hPL(1-28) suppression of IFN-gamma-inducible MHC class II antigens. FIG. 11B, hPL(1-28) suppression of IFN-gamma-inducible MHC class I antigens. FIG. 11 C, hPL(1-28) suppression of the IFN-gamma-inducible ICAM-1 antigen.

### DETAILED DESCRIPTION OF THE INVENTION

The invention pertains to interferon-suppressing placental lactogen peptides (ISPLP) which are derivable from naturally occurring amino acid sequences.

One aspect of the invention is an ISPLP comprising an isolated peptide with substantial sequence identity to hPL(1-28): VQTVPLSRLFDHAMLQAHRAHQLAIDTY (SEQ ID NO:4), wherein the peptide suppresses the action of IFN-gamma.

Another aspect of the invention is an ISPLP comprising an isolated peptide with substantial sequence identity to hPL-1(1-28): VQTVPLSRLFKEAMLQAHRAHQLAIDTY (SEQ ID NO:7), wherein the peptide suppresses the action of IFN-gamma.

In a further aspect, the invention conceives of derivatives of ISPLP. A derivative ISPLP is defined as a peptide derived from an ISPLP described above, which contains a linear portion of an ISPLP from 5 residues to 27 residues, which peptide suppresses the action of IFN-gamma. Said derivatives can be prepared in a manner similar to an ISPLP

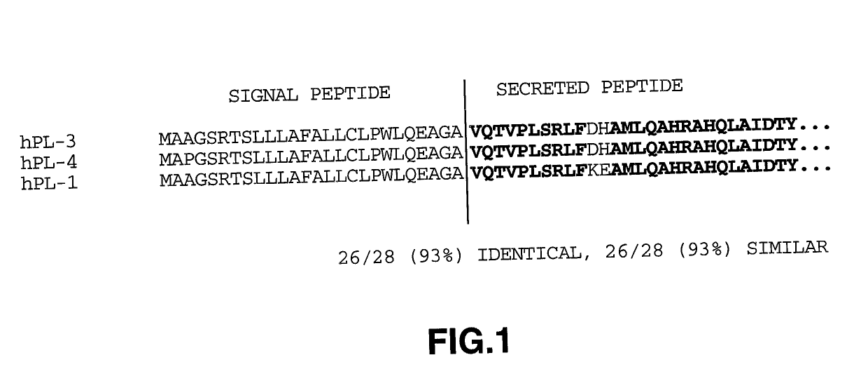

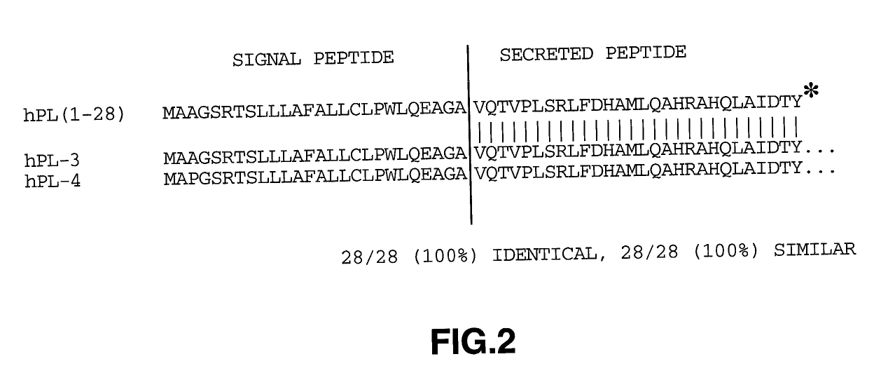

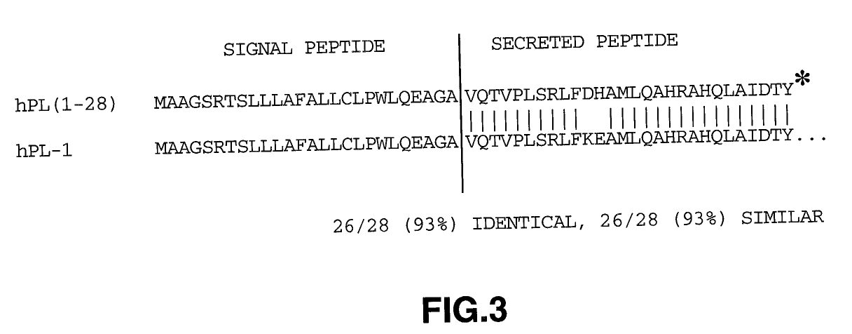

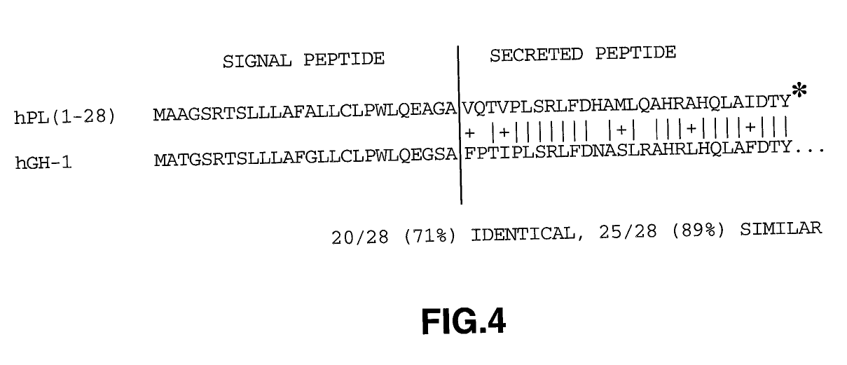

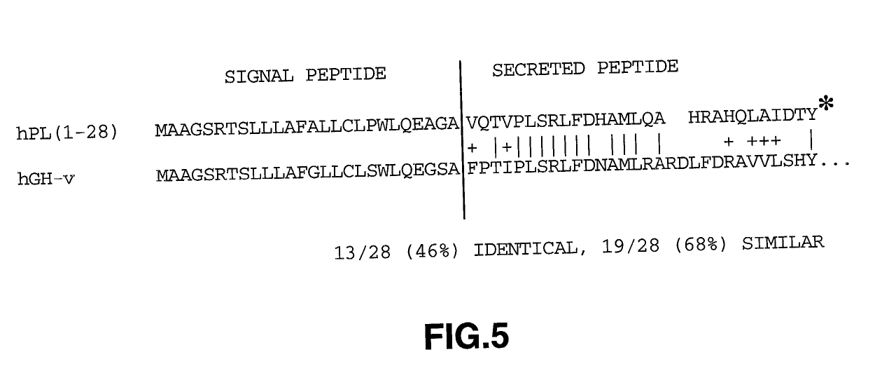

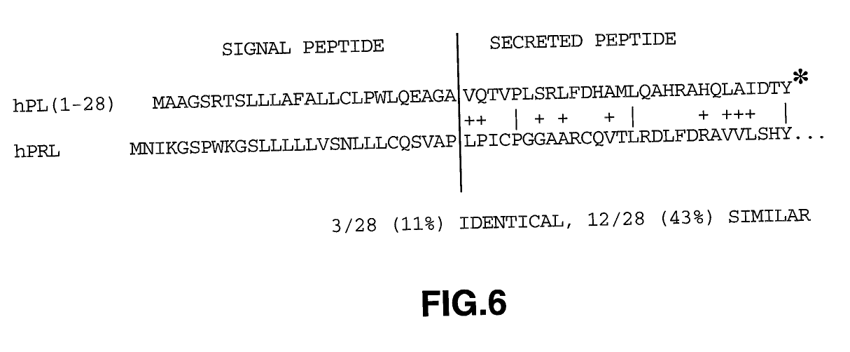

The human genetic studies cited above teach the loss of one or more, but not all, hPL hormones in subjects diagnosed with one of various pre-natal, intra-uterine, growth disorders, but no examples are shown which indicate that hGH-1 or hGH-V can replace hPL in prenatal human life. The studies cited also teach of negative correlations of hPL hormone levels with various defects of pregnancy. Consistent with the idea that the N-terminal domains of hPL hormones carry out functions not performed by hGH and hGH-V, including the suppression of IFN-gamma action, is the observation that the N-terminal 28 residue region of hPL is 93% conserved between the identical gene products of the hPL-3 and hPL-4 and the closely related gene product of the hPL-1 gene, as shown in FIGS. 1-3, but this 28 amino acid region is 71% identical in pituitary hGH-1 (FIG. 4) and only 46% identical in the placental hGH-V (FIG. 5). As well, hPL( 1-28) shares a mere 11% identity with hPRL (FIG. 6). Taken together, these facts provide strong evidence that the high abundance pregnancy hormones hPL-3, hPL-4 (herein called hPL), and the low abundance hormone hPL-1 act in concert to suppress IFN-gamma action, and have been maintained in the hGH/hPL locus on chromosome 17 in part to carry out that function. Conversely, the hGH-1, hGH-V, and hPRL hormones do not contain the highly conserved N-terminal domain motifs found in the hPL hormones which are shown herein to function as ISPLP. Accordingly, the invention is restricted to the hPL peptides of the hGH/hPL family, and excludes the corresponding, hGH peptides of the hGH/hPL family and hPRL.

The phrase “sequence with substantial identity” indicates that the peptide contains one or more conservative substitutions along the length its sequence, including substitutions from among the following 4 groups: hydrophobic and neutral amino acids (A, I, F, L, M, P, W, V), polar and neutral amino acids (C, G, N, Q, S, T, Y), basic amino acids (H,.R, K), and acidic amino acids (D, E). The invention encompasses all combinations of these substantially identical ISPLP, which can be prepared by methods that are routinely practised in the art, as described herein. The common characteristic inherent to each of these ISPLP is the functional activity defined as suppression of IFN-gamma action which is described herein and taught by example below.

TABLE 2
The single-letter codes for the amino acids

| | | |
|---|---|---|
| A | Alanine | Ala |
| C | Cysteine | Cys |
| D | Aspartic acid | Asp |
| E | Glutamic acid | Glu |
| F | Phenylalanine | Phe |
| G | Glycine | Gly |
| H | Histidine | His |
| I | Isoleucine | Ile |
| K | Lysine | Lys |
| L | Leucine | Leu |
| M | Methionine | Met |
| N | Asparagine | Asn |
| P | Proline | Pro |
| Q | Glutamine | Gln |
| R | Arginine | Arg |
| S | Serine | Ser |
| T | Threonine | Thr |
| V | Valine | Val |
| W | Tryptophan | Trp |
| Y | Tyrosine | Tyr |

Methods for Preparation of ISPLP from Cloned DNA Constructs

In one mode, the peptides of this invention are obtained by cloning the DNA sequence encoding the signal sequence and N-terminal (1-28) secreted peptide and stop codon into a vector, and transforming a host cell with the modified nucleic acid to allow expression and secretion of the peptide.

In a preferred mode, oligonucleotide-directed TA-cloning of peptide expression constructs is performed to produce ISPLP which is recovered from the culture medium after secretion from host cells. In oligo-directed TA-cloning, single-stranded oligodeoxynucleotides are designed that can anneal at their respective 3′-ends to form partially overlapping heteroduplexes which, when extended by a DNA polymerase, such as Taq polymerase or other DNA polymerase enzyme preparations known in the art, result in the generation of synthetic double-stranded DNA molecules encompassing the desired protein-coding capacity. The product of said extension, being double-stranded with single A-nucleotide overhangs on both 3′-ends, is ligated to a TA-cloning vector (e.g., pCR3.1, pcDNA4/HisMaxTOPO, pCRT7/VP22/TOPO) and cloned by methods known in the art. See for example, the Invitrogen Corp. Catalog (2001) for a thorough explanation of the practise of TA cloning. This reference provides descriptions of numerous variations of TA cloning including the use of vectors for expression of polypeptides in mammalian and other eukaryotic host cells, and the use of shuttle vectors for transferring expression constructs to several vectors for numerous purposes. Expression constructs such as this, as is commonly known in the art, may include, but not be restricted to, optional epitope tags and optional peptide tags and optional proteinase substrate sequences. The purposes of these optional sequences in a recombinant fusion protein include the facilitation of detection of the polypeptides in assays, and the facilitation of purification and characterization of the polypeptides, and the like. The Invitrogen Catalog provides detailed protocols for performing these procedures and information on how to select appropriate materials.

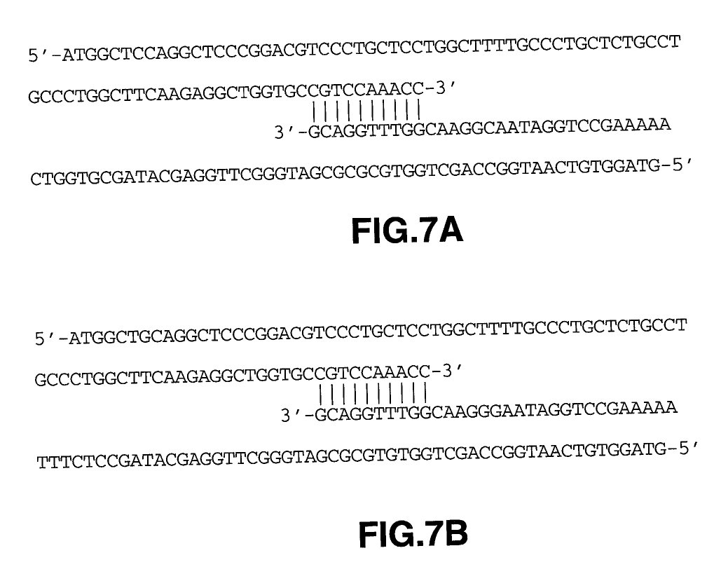

In a most preferred mode of the invention, the single-stranded oligodeoxynucleotides SEQ ID NO:8 and SEQ ID NO:10, shown schematically in FIG. 7A, are used in the oligo-directed TA-cloning of hPL(1-28). Cloning into pCR3.1 vector and over-expression of the secreted peptide in cultures of stably transfected human cells (e.g., HeLa) can provide serum-free conditioned medium which is used to purify the peptide. Methods of C-18 reverse-phase high-performance chromatography, using acetonitrile gradients with 0.1% trifluoroacetic acid counterion, can be used for the complete purification of the peptide from such mixtures. See below for details of peptide purification procedures.

Similarly, the single-stranded oligodeoxynucleotides SEQ ID NO:9 and SEQ ID NO:11, shown schematically in FIG. 7B, are used in the oligo-directed TA-cloning of hPL-1(1-28). The hPL-1 peptide can be expressed and purified like the hPL peptide above.

In another mode, an ISPLP is obtained by cloning the DNA sequence encoding an intact full-length human hormone into a vector, introducing a stop codon at the appropriate position by mutagenesis techniques known in the art; and transforming a host cell with the modified nucleic acid to allow expression of the encoded peptide.

In another mode, the ISPLP of the invention is produced by introducing into a bacterial host, such as E. Coli, a vector which drives the expression and secretion by the bacteria of the mature peptide without the mammalian signal sequence. Such bacterial expression systems are discussed below.

Methods for Expression of cDNAs Encoding Secreted Proteins in Bacteria

A wide range of single-cell and multicellular expression systems (i.e. host-expression vector combinations) can be used to produce the proteins of the invention. Possible types of host cells include, but are not limited to, bacterial, yeast, insect, mammalian, and the like. Many reviews are available which provide guidance for making choices and/or modifications of specific expression systems , e.g. to name a few, de Boer and Shepard, “Strategies for Optimizing Foreign Gene Expression in Escherichia coli,” pgs. 205-247, in Kroon, ed. Genes: Structure and Expression (John Wiley & Sons, New York, 1983), review several E. coli expression systems; Kucherlapati et al., CRC Crit RevBiochem 16: 4:349-379 (1984), and Banerji et al., Genetic Engineering 5:19-31 (1983) review methods for transfecting and transforming mammalian cells; Reznikoff and Gold, eds., Maximizing Gene Expression (Butterworths, Boston, 1986) review selected topics in gene expression in E. coli, yeast, and mammalian cells; and Thilly, Mammalian Cell Technology (Butterworths, Boston, 1986) reviews mammalian expression systems. Likewise, many reviews are available which describe techniques and conditions for linking and/or manipulating specific cDNAs and expression control sequences to create and/or modify expression vectors suitable for use with the present invention, e.g. Sambrook et al (cited above).

An E. coli expression system is disclosed by Riggs in U.S. Pat. No. 4,431,739, which is incorporated by reference. A particularly useful prokaryotic promoter for high expression in E. coli is the tac promoter, disclosed by de Boer in U.S. Pat. No. 4,551,433, which is incorporated herein by reference. Secretion expression vectors are also available for E. coli hosts. Preparations of appropriate DNA constructs encoding ISPLP without the native signal sequence can be made using methods that are known in the art. Particularly useful are the pIN-III-ompA vectors, disclosed by Ghrayeb et al. ((1984) EMBO J 3:2437-244), in which the cDNA to be transcribed is fused to the portion of the E. coli OmpA gene encoding the signal peptide of the ompA protein which, in turn, causes the mature protein to be secreted into the periplasmic space of the bacteria. U.S. Pat. Nos. 4,336,336 and 4,338,397 also disclose secretion expression vectors for prokaryotes. Accordingly, these publications and patents are incorporated by reference.

Numerous strains of bacteria are suitable hosts for prokaryotic expression vectors including strains of E. coli, such as W3110 (ATCC No. 27325), JA221, C600, ED767, DHI, LE392, HB101, X1776 (ATCC No. 31244), X2282, RRI (ATCC No. 31343) MRCI; strains of Bacillus subtilus; and other enterobacteria such as Salmonella typhimurium or Serratia marcescens, and various species of Pseudomonas. General methods for deriving bacterial strains, such as E. coli K12 X1776, useful in the expression of eukaryotic proteins is disclosed by Curtis III in U.S. Pat. No. 4,190,495. Accordingly this patent is incorporated by reference.

Methods for Expression of cDNAs Encoding Secreted Proteins in Mammalian Host Cells and Animals.

In addition to prokaryotic and eukaryotic microorganisms, expression systems comprising cells derived from multicellular organism may also be used to produce ISPLP of the invention. Of particular interest are mammalian expression systems because their post-translational processing machinery is more likely to produce biologically active mammalian proteins by proper cleavage of the signal sequence in ISPLP. Several DNA tumor viruses have been used as vectors for mammalian hosts. Particularly important are the numerous vectors which comprise SV40 replication, transcription, and/or translation control sequences coupled to bacterial replication control sequences, e.g. the pcD vectors developed by Okayama and Berg, disclosed in Mol Cell Biol 2:161-170 (1982) and Mol. Cell. Biol., Vol. 3, pgs. 280-289 (1983), and improved by Takebe et al, Mol Cell Biol 8:466-472 (1988). Accordingly, these references are incorporated herein by reference. Other SV40-based mammalian expression vectors include those disclosed by Kaufman and Sharp, in Mol Cell Biol 2:1304-1319 (1982), and Clark et al., in U.S. Pat. No. 4,675,285, both of which are incorporated herein by reference. Monkey cells are usually the preferred hosts for the above vectors. Such vectors containing the SV40 ori sequences and an intact A gene can replicate autonomously in monkey cells (to give higher copy numbers and/or more stable copy numbers than nonautonomously replicating plasmids). Moreover, vectors containing the SV40 ori sequences without an intact A gene can replicate autonomously to high copy numbers (but not stably) in COS7 monkey cells, described by Gluzman (Cell 23: 175-182 (1981) and available from the ATCC (accession no. CRL 1651). The above SV40-based vectors are also capable of transforming other mammalian cells, such as mouse L cells, by integration into the host cell DNA.

Multicellular organisms can also serve as hosts for the production of ISPLP, e.g. insect larvae, Maeda et al, (Nature 315: 592-594 (1985) and Ann Rev Entomol 351-372 (1989)); and transgenic animals, Jacnisch, Science 240: 1468-1474 (1988).

Methods for Preparation of ISPLP by Peptide Synthesis.

In another mode, the peptides may be prepared by peptide synthesis. Peptides of the invention are synthesized by standard techniques, e.g. Stewart and Young, Solid Phase Peptide Synthesis, 2nd Ed. (Pierce Chemical Company, Rockford, Ill., 1984). Preferably, a commercial peptide synthesizer is used, e.g. Applied Biosystems, Inc. (Foster City, Calif.) model 430A. Peptides of the invention are assembled by solid phase synthesis on a cross-linked polystyrene support starting from the carboxyl terminal residue and adding amino acids in a stepwise fashion until the entire peptide has been formed. The following references are guides to the chemistry employed during synthesis: Merrifield, J Amer Chem Soc 85: 2149 (1963); Kent et al., pg 185, in Peptides 1984, Ragnarsson, Ed. (Almquist and Weksell, Stockholm, 1984); Kent et al., pg. 217 in Peptide Chemistry 84, Izumiya, Ed. (Protein Research Foundation, B. H. Osaka, 1985); Merrifield, Science 232: 341-347 (1986); Kent, Ann. Rev. Biochem 57:957-989 (1988), and references cited in these latter two references.

In solid state synthesis it is most important to eliminate synthesis by-products, which are primarily termination, deletion, or modification peptides. Most side reactions can be eliminated or minimized by use of clean, well characterized resins, clean amino acid derivatives, clean solvents, and the selection of proper coupling and cleavage methods and reaction conditions, e.g. Barany and Merrifield, The Peptides, Cross and Meienhofer, Eds., Vol. 2, pgs 1-284 (Academic Press, New York, 1979). It is important to monitor coupling reactions to determine that they proceed to completion so that deletion peptides missing one or more residues will be avoided. The quantitative ninhydrin reaction is useful for that purpose, Sarin et al. (Anal Biochem 117:147 (1981)). Na-t-butyloxycarbonyl (t-Boc)-amino acids are used with appropriate side chain protecting groups stable to the conditions of chain assembly but labile to strong acids. After assembly of the protected peptide chain, the protecting groups are removed and the peptide anchoring bond is cleaved by the use of low then high concentrations of anhydrous hydrogen fluoride in the presence of a thioester scavenger, (Tam ct al., J Amer Chem Soc105:6442 (1983). Side chain protecting groups used are Asp(OBzl), Glu(OBzl), Scr(Bzl), Thr(Bzl), Lys(CI—Z), Tyr(Br—Z), Arg(NGTos), Cys(4-MeBzl), and His(ImDNP). (Bzl, benzyl; Tos, toluenesulfonyl; DNP, dinitrophenyl; Im, imidazole; Z, benzyloxgycarbonyl). The remaining amino acids have no side chain protecting groups. For each cycle the tBoc-Na protected peptide-resin is exposed to 65 percent trifluoroacetic acid (from Eastman Kodak) (distilled before use) in dichloromethane (DCM), (Mallenckrodt): first for 1 minute then for 13 minutes to remove the Na-protecting group. The peptide-resin is washed in DCM, neutralized twice with 10 percent diisopropylethylamine (DIEA) (Aldrich) in dimethylformarnide (DMF) (Applied Biosystems), for 1 minute each. Neutralization is followed by washing with DMF. Coupling is performed with the symmetric anhydride of the amino acid in DMF for 16 minutes. The symmetric anhydride is prepared on the synthesizer by dissolving 2 mmol of amino acid in 6 ml of DCM and adding 1 mmol of dicyclohexycarbodiimide (Aldrich) in 2 ml of DCM. After 5 minutes, the activated amino acid is transferred to a separate vessel and the DCM is evaporated by purging with a continuous stream of nitrogen gas. The DCM is replaced by DMF (6 ml total) at various stages during the purging. After the first coupling, the peptide-resin is washed with DCM, 10 percent DIEA in DCM, and then with DCM. For recoupling, the same amino acid and the activating agent, dicyclohexylcarbodiimide, are transferred sequentially to the reaction vessel. After activation in situ and coupling for 10 minutes, sufficient DMF is added to make a 50 percent DMF-DCM mixture, and the coupling is continued for 15 minutes. Arginine is coupled as a hydroxybenzotriazole (Aldrich) ester in DMF for 60 minutes and then recoupled in the same manner as the other amino acids. Asparagine and glutamine are coupled twice as hydroxybenzotriazole esters in DMF, 40 minutes for each coupling. For all residues, the resin is washed after the second coupling and a sample is automatically taken for monitoring residual uncoupled alpha.-amine by quantitative ninhydrin reaction, Sarin et al. (cited above).

Treatment of Immunological Disorders

In one mode of the invention, human cells or tissue are treated ex vivo with an effective amount of an ISPLP disclosed above and used for the treatment of subjects suffering from conditions exacerbated by the action of IFN-gamma, including, but not limited to, MHC-associated autoimmune disease. In a preferred mode, transplanted cells and tissues are treated, such as bone marrow, cornea, kidney, lung, liver, heart, skin, or pancreatic islets.

In another mode, subjects in need thereof are treated with effective amount of an ISPLP in vivo. Among the autoimmune diseases and inflammatory responses that can be treated (including treated prophylactically) with an ISPLP of the invention, optionally with a Type IV PDE inhibitor, are:

(a) autoimmune diseases, such as lupus erythematosus, multiple sclerosis, infertility from endometriosis, type I diabetes mellitus including the destruction of pancreatic islets leading to diabetes and the inflammatory consequences of diabetes, including leg ulcers, Crohn's disease, ulcerative colitis, inflammatory bowel disease, osteoporosis and rheumatoid arthritis;

(b) allergic diseases such as asthma, hay fever, rhinitis, vernal conjunctivitis and other eosinophil-mediated conditions;

(c) skin diseases such as psoriasis, contact dermatitis, eczema, infectious skin ulcers, open wounds, cellulitis;

(d) infectious diseases including sepsis, septic shock, encephalitis, infectious arthritis, endotoxic shock, gram negative shock, Jarisch-Herxheimer reaction, shingles, toxic shock, cerebral malaria, bacterial meningitis, acute respiratory distress syndrome (ARDS), lyme disease, HIV infection;

(e) wasting diseases: cachexia secondary to cancer and HIV;

(f) organ, tissue or cell transplantation (e.g., bone marrow, cornea, kidney, lung, liver, heart, skin, pancreatic islets) including transplant rejection, and graft versus host disease;

(g) adverse effects from drug therapy, including adverse effects from amphotericin B treatment, adverse effects from immunosuppressive therapy, e.g., interleukin-2 treatment, adverse effects from OKT3 treatment, adverse effects from GM-CSF treatment, adverse effects of cyclosporine treatment, and adverse effects of aminoglycoside treatment, stomatitis and mucositis due to immunosuppression;

(h) cardiovascular conditions including circulatory diseases induced or exasperated by an inflammatory response, such as ischemia, atherosclerosis, peripheral vascular disease, restenosis following angioplasty, inflammatory aortic aneurysm, vasculitis, stroke, spinal cord injury, congestive heart failure, hemorrhagic shock, ischemia/reperfusion injury, vasospasm following subarachnoid hemorrhage, vasospasm following cerebrovascular accident, pleuritis, pericarditis, and the cardiovascular complications of diabetes;

(i) dialysis, including pericarditis, due to peritoneal dialysis;

(j) gout; and

(k) chemical or thermal due to burns, acid, alkali and the like.

Purification and Pharmaceutical Compositions

When polypeptides of the present invention are expressed in soluble form, for example as a secreted product of transformed yeast or mammalian cells, they can be purified according to standard procedures of the art, including steps of ammonium sulfate precipitation, ion exchange chromatography, gel filtration, electrophoresis, affinity chromatography, and/or the like, e.g. “Enzyme Purification and Related Techniques,” Methods in Enzymology, 22:233-577 (1977), and Scopes, R., Protein Purification: Principles and Practice (Springer-Verlag, N.Y., 1982) provide guidance in such purifications. Likewise, when polypeptides of the invention are expressed in insoluble form, for example as aggregates, inclusion bodies, or the like, they can be purified by standard procedures in the art, including separating the inclusion bodies from disrupted host cells by centrifugation, solublizing the inclusion bodies with chaotropic and reducing agents, diluting the solubilized mixture, and lowering the concentration of chaotropic agent and reducing agent so that the polypeptide takes on a biologically active conformation. The latter procedures are disclosed in the following references, which are incorporated by reference: Winkler et al, Biochemistry 25: 4041-4045 (1986); Winkler et al, Biotechnology 3: 992-998 (1985); Koths et al, U.S. Pat. No. 4,569,790; and European patent applications 86/306917.5 and 86/306353.3.

As used herein “effective amount” means an amount sufficient to ameliorate a symptom of an autoimmune or inflammatory condition. The effective amount for a particular patient may vary depending on such factors as the state of the condition being treated, the overall health of the patient, method of administration, the severity of side-effects, and the like. Generally, ISPLP is administered as a pharmaceutical composition comprising an effective amount of ISPLP and a pharmaceutical carrier. A pharmaceutical carrier can be any compatible, non-toxic substance suitable for delivering the compositions of the invention to a patient. Generally, compositions useful for parenteral administration of such drugs are well known, e.g. Remington's Pharmaceutical Science, 15th Ed. (Mack Publishing Company, Easton, Pa. 1980). Alternatively, compositions of the invention may be introduced into a patient's body by implantable or injectable drug delivery system, e.g. Urquhart et al., Ann Rev Pharmacol Toxicol 24:199-236 (1984); Lewis, ed. Controlled Release of Pesticides and Pharmaceuticals (Plenum Press, N.Y., 1981); U.S. Pat. Nos. 3,773,919; 3,270,960; and the like.

When administered parenterally, the ISPLP is formulated in a unit dosage injectable form (solution, suspension, emulsion) in association with a pharmaceutical carrier. Examples of such carriers are normal saline, Ringer's solution, dextrose solution, and Hank's solution. Nonaqueous carriers such as fixed oils and ethyl oleate may also be used. A preferred carrier is 5% dextrose/saline. The carrier may contain minor amounts of additives such as substances that enhance isotonicity and chemical stability, e.g., buffers and preservatives. The ISPLP is preferably formulated in purified form substantially free of aggregates and other proteins. Preferably, ISPLP is administered by continuous infusion The daily infusion rate may be varied based on monitoring of side effects and immune status.

An appreciation of this aspect of the invention can be obtained through a consideration of having now fully described the invention, the same will be more readily understood by reference to specific examples which are provided by way of illustration, and are not intended to be limiting of the invention. Selection of vectors and hosts as well as the concentration of reagents, temperatures, and the values of other variable parameters are only to exemplify application of the present invention and are not to be considered as limitations thereof.

### Example 1.

Identification of hPL(1-28) in a Functional Screen for Placental Suppressors of IFN-gamma-Stimulated MHC Class II Expression in a Stably Transfected Reporter Cell Line.

Experimental Strategy

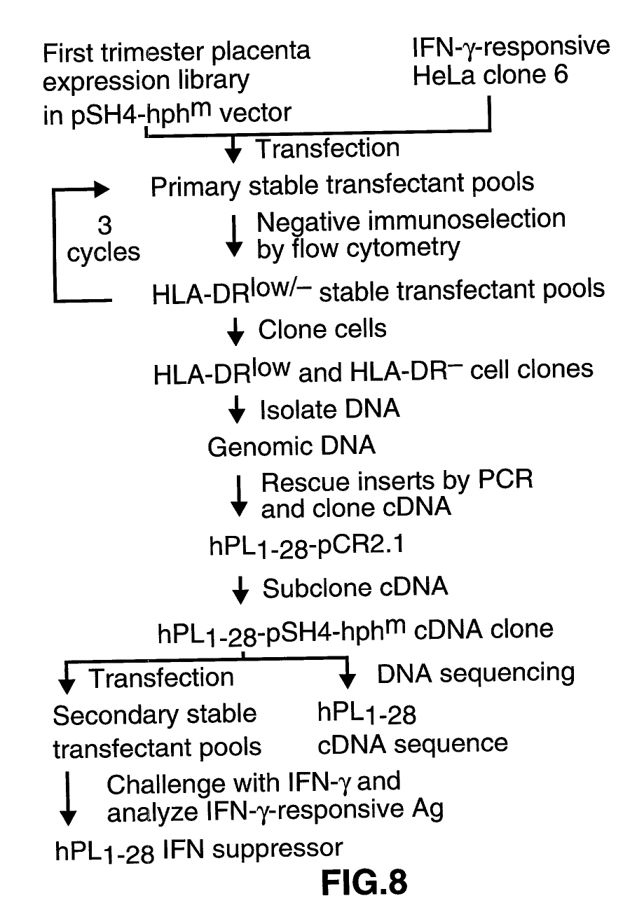

Mammalian expression cloning was used to test the hypothesis that trophoblasts express one or more dominant suppressors of IFN-gamma-induced expression of the MHC class II antigen HLA-DR. The expression cloning strategy obviated the need for structural information about the target cDNAs or the gene products encoded. This strategy was designed to detect by negative immunoselection those trophoblast cDNAs, in a stably transfected pool of human reporter cells, that encoded suppressor factors that blocked any step between the initial IFN-gamma binding and the final HLA-DR biosynthesis and delivery to the cell membrane. The method is presented schematically in FIG. 8.

Methods

A placenta sample was obtained from an elective termination of pregnancy at 10 weeks. PolyA+-RNA was prepared with the PolyATTract System (Promega). Placenta cDNAs were prepared with oligo-dT primers, size-selected, and cloned using standard methods (Sambrook J, Fritsch E F, Maniatis T: 1989. Molecular Cloning. A Laboratory Manual. Cold Spring Harbor, N.Y.: Cold Spring Harbor Laboratory Press) with directional adapters into the mammalian expression vector pSH4-hph.sup.m which provides an SV40 promoter, splice site upstream of the cloning site, and poly-A addition signal as well as hygromycin resistance (Vasavada H A et al. “pSH4: A mammalian expression vector.”). The cDNA library was grown in DH10B Electromax E. Coli (GIBCO), and the 3-dimensional amplification procedure in 50 ml tubes was carried out to ensure reasonable representation of slow growing bacteria (Kriegler M: 1990. Gene transfer and expression. A laboratory manual. Stockton Press, New York, N.Y.). Large plasmid preps with two CsCI bandings produced stock solutions for transfection. The first trimester placenta expression library consisted of 1.7×10 .sup.5 independent clones. A clone of the cervical carcinoma cell line HeLa (ATCC) was isolated by limiting dilution culture and expressed HLA-DR antigen after stimulation by recombinant human IFN-gamma (Boehringer-Mannheim). This HeLa clone 6 was expanded for further use. The cDNA expression library was transfected by the calcium phosphate method into HeLa clone 6 cells. A total of approximately 2×10. sup.4 stable transfectants resistant to 150.mu.g/ml hygromycin B (Boehringer-Mannheim) were screened from 4 transfections over several months. Three rounds of selection were performed by IFN-gamma challenge (200 U/ml for 2 days) and sterile sorting by flow cytometry of live, lightly trypsinized cells stained in suspension at 4° C., gating on the lowest 5-10% of the range of HLA-DR antigen staining. The HLA-DR mAb L243 (IgG2a (isotype, ATCC) and non-immune mouse IgG2a (Sigma) as negative control were used at 10. mu.g/ml. Untransformed, IFN-gamma-treated HeLa clone 6 cells served as positive control cells. mAb binding was detected with R-phycoerythrin-goat anti-mouse IgG secondary Ab (Molecular Probes) using a FACS IV (Becton-Dickinson). The isolation of antigen-negative cells was completed by cloning by limiting dilution and screening subcultures grown in chamber slides (Nunc) by immunocytochemistry using L243 mAb as described (Peyman J A and Hammond G L (1992) Localization of interferon-gamma receptor in first trimester placenta to trophoblasts but lack of stimulation of HLA-DRA, -DRB, or invariant chain MRNA expression by interferon-gamma. J Immunol 149: 2675-2680). Twelve cell clones resulted, and genomic DNA was prepared from each of these (Sambrook et al., 1989). Rescue of integrated plasmid sequences was accomplished by PCR (Innis M A, Gelfand D H, Sninsky J J, White, T J, eds: 1990. PCR protocols: A guide to methods and applications. Academic Press, San Diego, Calif). Primers were prepared that amplified sequences between the promoter and the poly-A signal of the expression vector, pSH4-1: 5′-GATGTTGCCTTTACTTCTAGGCCT-3′ (SEQ ID NO:12), and pSH4-2: 5′-AACTCATCAATGTATCTTATCATG-3′ (SEQ ID NO:13).

Amplification was performed over 30 cycles of 1 min at 94° C., 2 min at 55° C., and 3 min at 72° C. in a thermal cycler (Perkin Elmer) using 1U of Taq DNA polymerase (GIBCO) and 1ul of 67 pg/ml genomic DNA. PCR products were identified on agarose gels stained with 0.5. mu.g/nl ethidium bromide. PCR products were cloned after amplifying over 30 cycles of 0.5 min at 94° C., 1 min at 55° C., and 2 min at 72° C. in a thermal cycler (MJR), then gel purifying the 0.9 kb product, re-amplifying under the same conditions and ligating to the pCR2.1 vector (Invitrogen). Plasmids were purified from 4 bacterial clones using Qiaprep8 strips and Qiagen columns (Qiagen) for sequencing and subcloning.

Double-stranded plasmid DNA was sequenced by the fluorescent cycle sequencing method with an Applied Biosystems 373A DNA Sequencer. Primers used for sequencing were pSH4-1, pSH4-2, T7 promoter primer, and M13 reverse primer. Data were analyzed with the Genetics Computer Group programs (Devereux J, Haeberli P, Smithies O: A comprehensive set of sequence analysis programs for the VAX. Nucl Acids Res 1984; 12: 387-395), and searching of the combined sequence databases at NCBI was performed with the BLAST program (Altschul S F et al. (1990) J Mol Biol 215: 403-410 Basic local alignment search tool.).

Results

HeLa clone 6 cells were stably transfected with the placenta cDNA expression library. Those cells with reduced expression of IFN-gamma-stimulated HLA-DR antigen were selected by flow cytometry. Cells resulting from three rounds of sterile cell sorting were cloned by limiting dilution culture. A total of 12 cellular clones derived from 4 transfections were expanded for further analysis because they expressed low HLA-DR antigen levels on the cell membrane or in intracellular compartments when analyzed by avidin-biotin-peroxidase immunocytochemistry (results not shown).

Integrated cDNAs were rescued by polymerase chain reaction using flanking vector primers and genomic DNA prepared from the transfectant clones. Eight of the 12 cell clones gave rise to 0.9 kb PCR products (results not shown). The 0.9 kb PCR product from one cell clone was ligated to the pCR2.1 vector for sequencing.

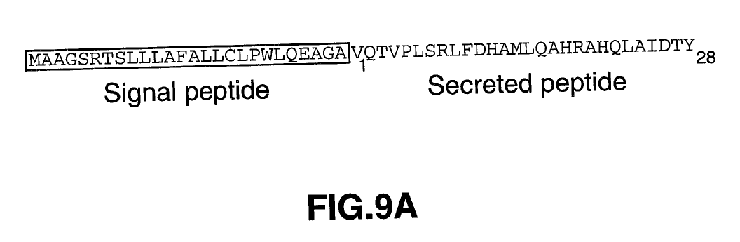

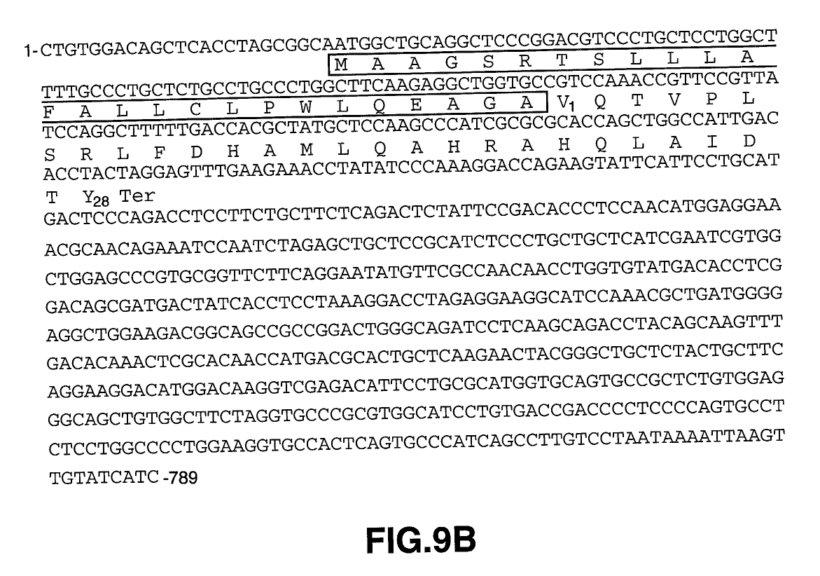

Plasmids from 4 bacterial clones of the 0.9 kb PCR product were sequenced using one of four pairs of vector primers. Sixteen overlapping sequences covering the insert eight times were obtained. The DNA and protein sequences are shown in FIG. 9. The signal sequence and a truncated N-terminal polypeptide of secreted hPL are encoded by this gene (FIG. 9A). Translation is terminated at a stop codon following residue 54, or residue 28 after cleavage of the signal sequence. This short gene product may have resulted from a mutation during the preparation of the cDNA library, during the extended period of in vivo selection of MHC-suppressed cell clones, or during the two PCR amplification steps carried out to rescue the integrated cDNAs from genomic DNA. This variant of hPL is not known to occur naturally (Goffin V et al. (1996) Endocrine Rev 17: 385-410 “Sequence-function relationships within the expanding family of prolactin, growth hormone, placental lactogen, and related proteins in mammals”). The sequences of the transfected genes in the other cell clones giving rise to the observed 0.9 kb PCR products remain to be determined.

Discussion

The isolation of a human placental lactogen clone as an interferon-gamma suppressor was unexpected. Specifically, a trophoblast cDNA was cloned that encoded the N-terminal 54 amino acids of hPL, including the 26 residue signal sequence and a 28 residue secreted peptide. An in-frame stop codon interrupted translation of the otherwise full-length hPL cDNA, and this gave rise to a fortuitous >85% deletion mutant of the hPL hormone.

### Example 2

Characterization of the Suppression of IFN-gamma-Stimulated Activities by hPL(1-28).

Experimental Strategy

The hPL( 1-28) cDNA was identified by the functional property of suppression of MHC class II antigen levels on the cell surface following IFN-gamma treatment, but this result alone could not rule out trivial mechanisms for this suppression such as insertional mutagenesis of one of the genes involved in IFN-gamma receptor signaling or in the post-translational processing of MHC class II component polypeptides. For this reason the function of the cloned cDNA was tested directly using pools of stably transfected HeLa cells. Expression of three families of endogenous IFN-gamma-responsive genes in this reporter cell line was determined by flow cytometry using monoclonal antibodies that recognize MHC class II antigens (HLA-DR, DP, and DQ alpha chains), MHC class I heavy chains (HLA-A, B, and C ), and ICAM-1 antigen.

Methods

The hPL(1-28)-encoding cDNA was subcloned into the mammalian expression vector pSH4-hphm using standard methods. HeLa cells (100 mm dishes) were transfected with 4 mg of HPL(1-28)-pSH4-hph.sup.m DNA or empty vector (negative control) by calcium phosphate-mediated transfection. Stable transfectants were selected with hygromycin at 10 mg/ml for at least 16 days. Flow cytometric analysis was performed on stably transfected pools of cells that were treated in culture with IFN-gamma (0, 20, 66, 200, and 1000 U/ml) for 42 h and suspended for analysis by light trypsinization. Cells were stained at 4° C. with 10 .mu.g/ml solutions of mAb CR3/43 (Dako) specific for MHC class II antigens HLA-DP, HLA-DQ, and HLA-DR, mAb G46-2.6 (Pharmingen) specific for MHC class I antigens HLA-A, HLA-B, and HLA-C heavy chains, mAb HA58 (Pharmingen) specific for ICAM-1 antigen, and either non-immune mouse IgG1 or IgG2a as negative control. Antibody binding was detected with FITC-goat anti-mouse antibody (Biomeda), and 20,000 cells were analyzed with a FACScan (Becton-Dickinson).

Results

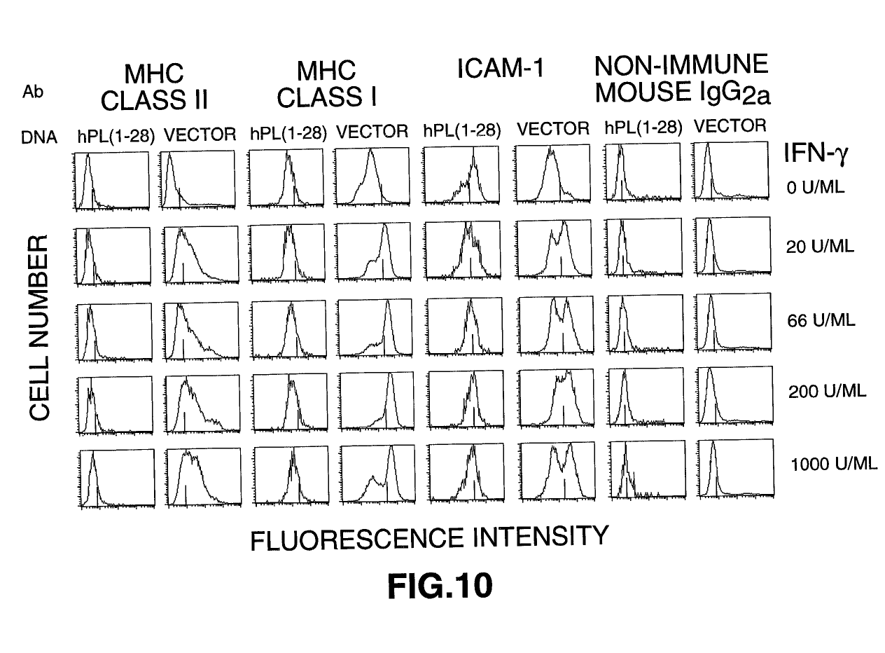

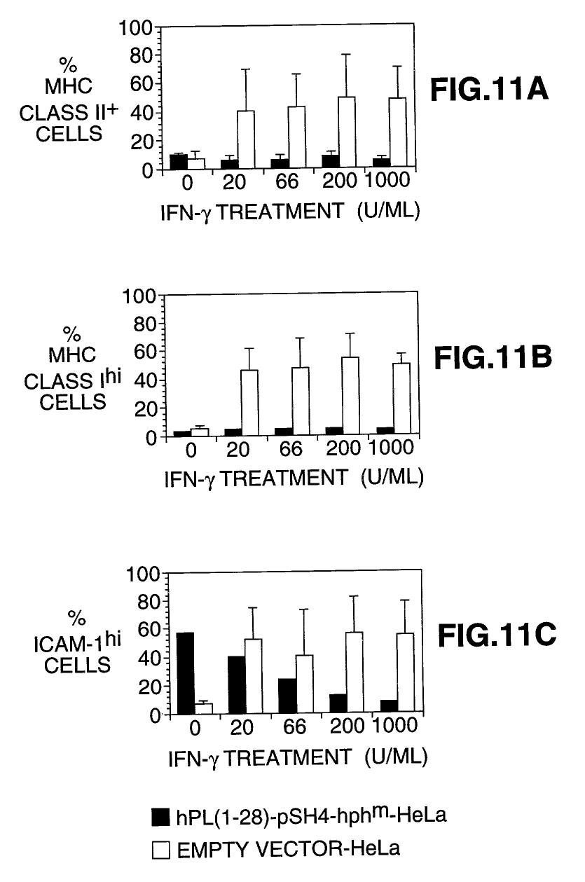

FIG. 10 shows the results from an experiment with the three antibodies and hPL(1-28)-expressing cells. Mean values are represented graphically in FIG. 11 (MHC class II, n=2; corresponding control cells, n=3; MHC class I, n=1; corresponding control cells, n=3; ICAM-1, n=1; corresponding control cells, n=3). Error bars represent standard deviations. The levels of HLA-DR, DP, and DQ antigens were not increased by treatment of the hPL( 1-28)-expressing cells at any dose of IFN-gamma tested up to 1000 U/ml (FIG. 10, and FIG. 11A filled bars). Vector-transfected control cells responded to IFN-gamma treatment with an increase in HLA-DR, DP, and DQ antigens (FIG. 10, and FIG. 11A open bars) HLA-A, B, and C antigens were expressed at moderate levels above background in the absence of IFN-gamma treatment in the hPL( 1-28)-expressing cells and in the control transfectants (FIG. 10). That is, the constitutive expression of MHC class I genes was not reduced in cells expressing hPL(1-28), but IFN-gamma treatment did not induce high level expression of class I genes in these cells (FIG. 10, and FIG. 11B filled bars). Vector-transfected control cells responded to IFN-gamma treatment with high levels of membrane HLA-A, B, and C antigens (FIG. 10, and FIG. 11B open bars). More than 50% of cells expressed ICAM-1 at high level in the absence of IFN-gamma treatment in the hPL(1-28)-expressing cells (FIG. 10, and FIG. 11C filled bars). The fraction of ICAM-1 high-level positive cells was less in the hPL(1-28)-expressing cells treated with 20 U/ml of IFN-gamma and still lower in cell treated with higher doses of cytokine (FIG. 10, and FIG. 11C filled bars).

Discussion

The hPL(1-28)-encoding cDNA was used to show suppression of IFN-gamma-induced MHC class I and class II and ICAM-1 expression in stably transfected HeLa cells expressing hPL(1-28). The mechanism of this block in IFN-gamma signaling or subsequent biosynthetic steps is not known. The Jak-STAT pathway may be involved since hPL has been shown to cause activation and phosphorylation of STAT3 and other undefined proteins in human MCF7 cells in culture (Takeda T, Kurachi H, Yamamoto T, Homma H, Morishige K, Miyake A, Murata Y: Participation of JAK, STAT and unknown proteins in human placental lactogen-induced signaling: a unique signaling pathway different from prolactin and growth hormone. J Endocrinol 1997; 153: R1-3). The possible MHC-suppressive functions of STAT3 and their relationship to the MHC-inducing functions of STAT1 have not been investigated. Alternatively, other cell signaling or biosynthetic pathways may be blocked by the effects of hPL(1-28) on HeLa cells. In this work seven other cell clones from several transfections with the placental expression library yielded PCR products using genomic DNA and vector primers that resembled the 0.9 kb size of this hPL cDNA, and these PCR products remain to be cloned and sequenced. The fact that hPL-encoding mRNA is perhaps the most abundant message produced by trophoblasts, and the fact that one of the activities of hPL is the suppression of IFN-gamma action, when taken together, are consistent with the observation of multiple immunosuppressed cell clones containing transfected cDNAs resembling hPL in size.

The descriptions of the foregoing embodiments of the invention have been presented for purpose of illustration and description. They are not intended to be exhaustive or to limit the invention to the precise forms disclosed, and obviously many modifications and variations are possible in light of the above teaching. The embodiments were chosen and described in order to best explain the principles of the invention to thereby enable others skilled in the art to best utilize the invention in various embodiments and with various modifications as are suited to the particular use contemplated. It is intended that the scope of the invention be defined by the claims appended hereto.

References

The papers and patents cited herein are expressly incorporated in the entireties by reference.

Gerhart J. and Kirschner M. (1997) Cells, embryos, and evolution: Toward a cellular and developmental understanding of phenotypic variation and evolutionary adaptability. Blackwell Science (Malden,Mass.).

Hunt J S and Orr H T (1992) FASEB J 6: 2344-2348 “HLA and maternal-fetal recognition.”

Cross J C et al. (1994) Science 266: 1508-1518 “Implantation and the placenta: Key pieces of the development puzzle.”

Wood G W (1994) Immunol Today 15: 15-18 “Is antigen presentation the explanation for fetal allograft survival?”

Peyman J A and Hammond G L (1992) J Immunol 149: 2675-80 “Localization of interferon-gamma receptor in first trimester placenta to trophoblasts but lack of stimulation of HLA-DRA, -DRB, or invariant chain mRNA expression by interferon-gamma.”

Peyman J A (1999) Biol Reprod 60: 23-31 “Repression of major histocompatibility complex genes by a human trophoblast ribonucleic acid.”

Ting J P and Zhu X S (1999) Microbes Infect 1:855-61 “Class II MHC genes: a model gene regulatory system with great biologic consequences.”

Van Den Elsen P J et al. (1998) Immunogenetics 48:208-21 “Regulation of MHC class I and II gene transcription: differences and similarities.”

Piskurich J F et al. (1999) Mol Cell Biol 19:431-440 “Two distinct gamma interferon-inducible promoters of the major histocompatibility complex class II transactivator gene are differentially regulated by STAT1, interferon regulatory factor 1, and transforming growth factor β”

Hooks et al. (1979), New England J Med 301: 5-8.

Battazzoetal. (1983) Lancet 1115-1119.

Hooks et al. (1980) Ann NY Acad Sci 21-32.

Iwatani et al (1986) J Clin Endocrin and Metabol 63:695-708.

Lider et al., (1988) Science 239:181 “Anti-idiotypic network induced by T cell vaccination against experimental autoimmune encephalomyelitis.”

Owhashiand et. al. (1988) J Exp Med 168:2153 “Protection from experimental allergic encephalomyelitis conferred by a monoclonal antibody directed against a shared idiotype on rat T cell receptors specific for myelin basic protein.”

Brostoffand et al. (1984) J Immunol 133:1938 “Experimental allergic encephalomyelitis: successful treatment in vivo with a monoclonal antibody that recognizes T helper cells.”

Adorini et al. (1988) Nature 334:623-628 “Dissociation of phosphoinositide hydrolysis and Ca2+ fluxes from the biological responses of a T-cell hybridoma.”

Chen E Y et al. (1989) Genornics 4 (4), 479-497 “The human growth hormone locus: nucleotide sequence, biology, and evolution.”

Misra-Press. A et al. (1994) J. Biol Chem 269:23220-23229 “Complex alternative splicing partially inactivates the human chorionic somatomammotropin-like (hCS-L) gene.”

Gossens et al. (1986) J. Clin Endo Metab 62: 712-716.

Wurzel et al. (1982) DNA 1: 251-257.

Sambrook J Fritsch E F Maniatis T (1989) Molecular Cloning: A Laboratory Manual (Cold Spring Harbor, N.Y.) Cold Spring Harbor Laboratory Press.

Vasavada H A et al. (1990) Nucl Acids Res 18: 3668-3668 “pSH4: A mammalian expression vector.”

Kriegler M (1990) Gene transfer and expression. A laboratory manual. Stockton Press, New York, N.Y.

Innis M A et al. eds: 1990. PCR protocols: A guide to methods and applications. (Academic Press, San Diego, Calif.).

Devereux J et al. (1984) Nucl Acids Res 12: 387-395 “A comprehensive set of sequence analysis programs for the VAX.”

Altschul S F et al. (1990) J Mol Biol 215: 403-410 “Basic local alignment search tool.”

Goffin V et al. (1996) Endocrine Rev 17: 385-410 “Sequence-function relationships within the expanding family of prolactin, growth hormone, placental lactogen, and related proteins in mammals.”

Takeda T et al. (1997) J Endocrinol 153: R1-3 “Participation of JAK, STAT and unknown proteins in human placental lactogen-induced signaling: a unique signaling pathway different from prolactin and growth hormone.”

Stephenson M D (1996) Fertil Steril 66: 24-29 “Frequency of factors associated with habitual abortion in 197 couples.” Forsyth I A (1994) Exp Clin Endocrinol 102: 244-251 “Comparative aspects of placental lactogens: structure and function.”

Byatt J C et al. 1992) J Animal Science 70: 2911-2913 “Ruminant placental lactogens: structure and biology.”

Byatt J C (1994)J Endocrinol 140: 33-43 “Stimulation of mammogenesis and lactogenesis by recombinant bovine placental lactogen in steroid-primed dairy heifers.”

Bridges R S and Freemark MS (1995) Hormones & Behavior 29: 216-226 “Human placental lactogen infusions into the medial preoptic area stimulate maternal behavior in steroid-primed, nulliparous female rats.”

Freemark M (1989) Endocrinol 125: 1504-1512 “Nutritional regulation of the placental lactogen receptor in fetal liver: implications for fetal metabolism and growth.”

Wallis M (1996) J Mol Evol 43: 93-100 “The molecular evolution of vertebrate growth hormones: a pattern of near-stasis interrupted by sustained bursts of rapid change.”

Forsyth I A (1994) Exp Clin Endocrinol 102: 244-245 “Comparative aspects of placental lactogens: structure and function.”

Golos T G et al. (1993) Endocrinol 133: 1744-1752 “Cloning of four growth hormone/chorionic somatomammotropin-related complementary deoxyribonucleic acids differentially expressed during pregnancy in the rhesus monkey placenta.”

Forsyth I A (1986) J Dairy Sci 69: 886-903 “Variation among species in the endocrine control of mammary growth and function: the roles of prolactin, growth hormone, and placental lactogen.”

Cunningham B C and Wells J A (1989) Science 244: 1081-1085 “High-resolution epitope mapping of hGH-receptor interactions by alanine-scanning mutagenesis.”

Luck D N et al. (1989) Mol Endocrinol 3: 822-823 “Bioactive recombinant methionyl bovine prolactin: structure-function studies using site-specific mutagenesis.”

Scott P et al. (1992) Mol Cell Endocrinol 89: 47-58 “Molecular cloning of the bovine prolactin receptor and distribution of prolactin and growth hormone receptor transcripts in fetal and utero-placental tissues.”

Gertler A et al. (1992) J Biol Chem 267: 12655-12659 “Preparation, purification, and determination of the biological activities of 12 N terminus-truncated recombinant analogues of bovine placental lactogen.”

Lowman H B et al. (1991) J Biol Chem 266: 10982-10988 “Mutational analysis and protein engineering of receptor-binding determinants in human placental lactogen.”

Binder L et al. (1990) Mol Endocrinol 19904: 1060-1068 “Site-directed mutations of human growth hormone that selectively modify its lactogenic activity and binding properties.”

Freemark M et al. (1987) Endocrinol 120: 1865-1872 “A unique placental lactogen receptor: implications for fetal growth.”

Ormandy C J (1997) Genes Dev 11:167-78 “Null mutation of the prolactin receptor gene produces multiple reproductive defects in the mouse.”

Gosseye S and Van Der Veen F (1992) Eur J Obst Gyn Reprod Biol 44: 85-90 “HPL-positive infiltrating trophoblastic cells in normal and abnormal pregnancy.”

Redline R W and Patterson P (1995) Hum Pathol 26: 594-600 “Pre-eclampsia is associated with an excess of proliferative immature intermediate trophoblast.”

Strasburger C J et al. (1989) Mol Cell Endocrinol 67: 55-62 “Indication of different lactogen and somatogen binding sites in the human growth hormone molecule as probed with monoclonal antibodies.”

Invitrogen Corp. Catalog, 2001.

Contractor S F and Davies H (1973) Nature 243: 284 “Effect of human chorionic somatomammotropin and human chorionic gonadotrophin on phytohemagglutinin-induced lymphocyte transformation.”

Cerni C et al. (1977) Arch Gynak 223: 1-7 “Immunosuppression by human placental lactogen (HPL) and the pregnancy-specific betal-glycoprotein (SP-1)”.

Hammerstrom L et al. (1979) Acta Obstet Gynecol Scand 58: 417-422 “The immunodepressive effect of human glucoproteins and their possible role in the nonrejection process during pregnancy.”

Ng FM et al. (1974) Diabetes 23:943-949).

Ng FM (1988) Diabetes Res Clin Pract 19:17-24 “A comparison of cellular actions between gliclazide and a hypoglycaemic peptide fragment of human growth hormone.”

Struman I et al. (1999) Proc Nati Acad Sci USA 96: 246-1251 “Opposing actions of intact and N-terminal fragments of the human prolactin/growth hormone family members on angiogenesis: An efficient mechanism for the regulation of angiogenesis.”

De Boer and Shepard, “Strategies for Optimizing Foreign Gene Expression in Escherichia coli,” pgs. 205-247,in Kroon, ed. Gene: Structure and Expression(John Wiley & Sons, New York, 1983).

Kucherlapati et al. (1984) CRC Crit Rev Biocheml6:349-379. Banerji et al. (1983) Genetic Eng. 5:19-31

Reznikoff and Gold, eds., Maximizing Gene Expression (Butterworths, Boston, 1986).

Thilly, Mammalian Cell Technology (Butterworths, Boston, 1986).

U.S. Pat. No. 4,431,739

U.S. Pat. No. 4,551,433

Ghrayeb et al.(1984) EMBO J 3:2437-2442.

U.S. Pat. Nos. 4,336,336.

U.S. Pat. Nos. 4,338,397.

U.S. Pat. No. 4,190,495

U.S. Pat. No. 4,675,285.

Okayama and Berg (1982) Mol Cell Biol 2:161-170.

Okayama and Berg (1983) Mol Cell Biol 3:280-289.

Takebe et al. (1988) Mol Cell Biol 8:466-472.

Kaufman and Sharp (1982) Mol Cell Biol 2:1304-1319.

U.S. Pat. No. 5,506,107.

U.S. Pat. No. 4,363,877.

U.S. Pat. No. 5,955,346.

U.S. Pat. No. 6,013,478.

U.S. Pat. No. 5,580,723.

U.S. Pat. No. 5,534,617.

WO 98/51323.

1. An interferon-suppressing placental lactogen peptide (ISPLP) comprising a sequence of amino acids selected from the group consisting of:
(a) the N-terminal 28 residues of hPL, VQTVPLSRLFDHAMLQAHRAHQLAIDTY (seq id no:4),
(b) a 28 amino acid sequence with substantial identity to seq id no:4, containing one or more conservative amino acid substitutions,
(c) a derivative of seq id no:4, comprising from 5 to 27 residues, and
(d) a derivative of the 28 amino acid sequence with substantial identity to seq id no:4, comprising from 5 to 27 residues;
which peptide suppresses IFN-gamma-stimulated expression of MHC class II antigens, MHC class I antigens, and ICAM-1 antigen:

2. An interferon-suppressing placental lactogen peptide (ISPLP) comprising a sequence of amino acids selected from the group consisting of:
(a) the N-terminal 28 residues of hPL-1, VQTVPLSRLFKEAMLQAHRAHQLAIDTY (SEQ ID NO:7),
(b) a 28 amino acid sequence with substantial identity to SEQ ID NO:7, containing one or more conservative amino acid substitutions,
(c) a derivative of SEQ ID NO:7, comprising from 5 to 27 residues, and
(d) a derivative of the 28 amino acid sequence with substantial identity to SEQ ID NO:7, comprising from 5 to 27 residues;
which peptide suppresses IFN-gamma-stimulated expression of MHC class II antigens, MHC class I antigens, and ICAM-1 antigen.

3. A peptide according to claim 1, which suppresses IFN-gamma-stimulated expression of an MHC class II antigen.

4. A peptide according to claim 1, which suppresses IFN-gamma-stimulated expression of an MHC class I antigen.

5. A peptide according to claim 1, which suppresses IFN-gamma-stimulated expression of ICAM-1 antigen.

6. A peptide according to claim 2, which suppresses IFN-gamma-stimulated expression of an MHC class II antigen.

7. A peptide according to claim 2, which suppresses IFN-gamma-stimulated expression of an MHC class I antigen.

8. A peptide according to claim 2, which suppresses IFN-gamma-stimulated expression of ICAM-1 antigen.

9. An interferon-suppressing placental lactogen peptide (ISPLP), having the sequence VQTVPLSRLFDHAMLQAHRAHQLAIDTY (SEQ ID NO:4), which suppresses IFN-gamma-stimulated expression of an MHC class 1I antigen.

10. An interferon-suppressing placental lactogen peptide (ISPLP), having the sequence VQTVPLSRLFKEAMLQAHRAHQLAIDTY (SEQ ID NO:7), which suppresses IFN-gamma-stimulated expression of an MHC class II antigen.

11. A method for treating a human subject in need thereof, comprising administering to the subject an effective amount of a cell or tissue that has been treated ex vivo with a peptide according to claim 1.

12. A method for treating a human subject in need thereof, comprising administering to the subject an effective amount of a cell or tissue that has been treated ex vivo with a peptide according to claim 2.

13. A method for treating a human subject in need thereof, comprising administering to the subject an effective amount of a cell or tissue that has been treated ex vivo with a peptide according to claim 9.

14. A method for treating a human subject in need thereof, comprising administering to the subject an effective amount of a cell or tissue that has been treated ex vivo with a peptide according to claim 10.

15. A method of treating a human subject presenting with autoimmune disease, inflammatory disease, or organ transplant rejection comprising administering to the subject an effective amount of an ISPLP of claim 1.

16. A method of treating a human subject presenting with autoimmune disease, inflammatory disease, or organ transplant rejection comprising administering to the subject an effective amount of an ISPLP of claim 2.

17. A method of treating a human subject presenting with autoimmune disease, inflammatory disease, or organ transplant rejection comprising administering to the subject an effective amount of an ISPLP of claim 9.

18. A method of treating a human subject presenting with autoimmune disease, inflammatory disease, or organ transplant rejection comprising administering to the subject an effective amount of an ISPLP of claim 10.


SEQUENCE LISTING (16 sequences)

SEQ ID NO: 1 - DNA, 789 bp, HOMO SAPIENS
  hPL(1-28) cDNA
ctgtggacagctcacctagcggcaatggctgcaggctcccggacgtccctgctcctggcttttgccctgctctgcctgccctggcttcaagaggctggtgccgtccaaaccgttccgttatccaggctttttgaccacgctatgctccaagcccatcgcgcgcaccagctggccattgacacctactaggagtttgaagaaacctatatcccaaaggaccagaagtattcattcctgcatgactcccagacctccttctgcttctcagactctattccgacaccctccaacatggaggaaacgcaacagaaatccaatctagagctgctccgcatctccctgctgctcatcgaatcgtggctggagcccgtgcggttcttcaggaatatgttcgccaacaacctggtgtatgacacctcggacagcgatgactatcacctcctaaaggacctagaggaaggcatccaaacgctgatggggaggctggaagacggcagccgccggactgggcagatcctcaagcagacctacagcaagtttgacacaaactcgcacaaccatgacgcactgctcaagaactacgggctgctctactgcttcaggaaggacatggacaaggtcgagacattcctgcgcatggtgcagtgccgctctgtggagggcagctgtggcttctaggtgcccgcgtggcatcctgtgaccgacccctccccagtgcctctcctggcccctggaaggtgccactcagtgcccatcagccttgtcctaataaaattaagttgtatcatc

SEQ ID NO: 2 - protein, 54 aa, HOMO SAPIENS
  hPL(1-28) signal sequence and secreted peptide, or N-terminal 54 residues of hPL-3
MAAGSRTSLLLAFALLCLPWLQEAGAVQTVPLSRLFDHAMLQAHRAHQLAIDTY

SEQ ID NO: 3 - protein, 54 aa, HOMO SAPIENS
  N-terminal 54 residues of hPL-4
MAPGSRTSLLLAFALLCLPWLQEAGAVQTVPLSRLFDHAMLQAHRAHQLAIDTY

SEQ ID NO: 4 - protein, 28 aa, HOMO SAPIENS
  hPL(1-28) peptide
VQTVPLSRLFKEAMLQAHRAHQLAIDTY

SEQ ID NO: 5 - DNA, 162 bp, HOMO SAPIENS
  cDNA construct coding for signal sequence and secreted peptide of hPL(1-28)
atggctccaggctcccggacgtccctgctcctggcttttgccctgctctgcctgccctggcttcaagaggctggtgccgtccaaaccgttccgttatccaggctttttgaccacgctatgctccaagcccatcgcgcgcaccagctggccattgacacctac

SEQ ID NO: 6 - protein, 54 aa, HOMO SAPIENS
  N-terminal 54 residues of hPL-4
MAAGSRTSLLLAFALLCLPWLQEAGAVQTVPLSRLFKEAMLQAHRAHQLAIDTY

SEQ ID NO: 7 - protein, 28 aa, HOMO SAPIENS
  hPL-1(1-28) peptide
VQTVPLSRLFKEAMLQAHRAHQLAIDTY

SEQ ID NO: 8 - DNA, 162 bp, HOMO SAPIENS
  cDNA construct coding for signal sequence and secreted peptide of hPL-1(1-28)
atggctgcaggctcccggacgtccctgctcctggcttttgccctgctctgcctgccctggcttcaagaggctggtgccgtccaaaccgttcccttatccaggctttttaaagaggctatgctccaagcccatcgcgcacaccagctggccattgacacctac

SEQ ID NO: 9 - DNA, 87 bp, HOMO SAPIENS
  hPL(1-28) 87-mer oligo
atggctccaggctcccggacgtccctgctcctggcttttgccctgctctgcctgccctggcttcaagaggctggtgccgtccaaacc

SEQ ID NO: 10 - DNA, 85 bp, HOMO SAPIENS
  hPL(1-28) 85-mer oligo
gtaggtgtcaatggccagctggtgcgcgcgatgggcttggagcatagcgtggtcaaaaagcctggataacggaacggtttggacg

SEQ ID NO: 11 - DNA, 85 bp, HOMO SAPIENS
  hPL-1(1-28) 85-mer oligo
gtaggtgtcaatggccagctggtgtgcgcgatgggcttggagcatagcctctttaaaaagcctggataagggaacggtttggacg

SEQ ID NO: 12 - DNA, 24 bp, ARTIFICIAL SEQUENCE
  pSH4-1
gatgttgcctttacttctaggcct

SEQ ID NO: 13 - DNA, 24 bp, ARTIFICIAL SEQUENCE
  pSH4-2
aactcatcaatgtatcttatcatg

SEQ ID NO: 14 - protein, 54 aa, HOMO SAPIENS
  N-TERMINAL 54 RESIDUES OF HGH-1
MATGSRTSLLLAFGLLCLPWLQEGSAFPTIPLSRLFDNASLRAHRLHQLAFDTY

SEQ ID NO: 15 - protein, 56 aa, HOMO SAPIENS
  N-TERMINAL 56 RESIDUES OF HGH-V
MAAGSRTSLLLAFGLLCLSWLQEGSAFPTIPLSRLFDNASLRARDLFDRAVVLSHY

SEQ ID NO: 16 - protein, 56 aa, HOMO SAPIENS
  N-TERMINAL 56 RESIDUES OF HPRL
MNIKGSPWKGSLLLLLVSNLLLCQSVAPLPICPGGAARCQVTLRDLFDRAVVLSHY